(19) United States
(12) Patent Application Publication
(10) Pub. No.: US 2009/0093885 A1
(43) Pub. Date: Apr. 9, 2009

(54) ANATOMIC INTERVERTEBRAL SPACER AND THE APPLICATIONS THEREOF

(75) Inventors: Jerome Levieux, Bellevue (CH); Patrick Tropiano, Marseille (FR)

Correspondence Address: YOUNG & THOMPSON, 209 Madison Street, Suite 500, ALEXANDRIA, VA, 22314, US

(73) Assignee: SPINEART S.A., MEYRIN GENEVE (CH)

(21) Appl. No.: 12/158,064

(22) PCT Filed: Dec. 19, 2006
(86) PCT No.: PCT/FR2006/002792

§ 371 Date: Oct. 30, 2008

(30) Foreign Application Priority Data
Dec. 20, 2005 (FR) 0512979

(51) Int. Cl. A61F 2/44

(52) U.S. Cl. 623/17.16; 623/17.11

(57) ABSTRACT

A multi-layer intervertebral spacer (1) includes at least three superimposed layers, i.e. lower (3), top (2) and at least one intermediate (4) layers, wherein the at least one intermediate layer (4) is made of a material which differs from the material of the lower (3) and top (2) layers and is less rigid and the inventive spacer, in a view from below, is generally shaped in the form of a bean and can be placed by means of a posterior or pasterolateral surgery.

The present invention relates to an anatomical intervertebral spacer and the applications thereof.

Lumbar spinal pathologies are currently treated in different ways.

Arthrodeses consist of locking a disk in order to prevent movement between two adjacent vertebrae. They are carried out using cages introduced between two vertebral bodies. The cage is a kind of solid box, the centre of which is filled with spongy bone. The walls of the cages are open-worked to allow fusion with the vertebral bodies. Such cages are for example described in FR-A-2 851 457, 2 736 538, 2 703 580, 2 816 201, 2 808 673 and 2 790 945. But they limit the patient's range of movement. In addition, they increase the work of the adjacent disks, which contributes towards their quicker wear.

Anterior lumbar arthroplasties by disk prosthesis represent an alternative which remedies the drawbacks of arthrodesis. Here, prostheses are used to replace the disk and are constituted by two plates in contact with the vertebral bodies, placing between them an intermediate piece which is either a sphere or a piece of elastic material. In the case of the system using a sphere (termed ball-socket) the upper plate on its internal face has a spherical shape congruent with the sphere of the intermediate piece in order to obtain natural movements of the vertebral column. In the case of an intermediate piece made of elastic material the movements are reproduced by deformation.

These recently used techniques have the advantage of restoring the disk movements. But their implantation can be tricky, even dangerous. Indeed the anterior implantation of prostheses requires the mobilization of the aorta and the vena cava, which can be dangerous. Moreover, many spinal column surgeons are not used to anterior techniques, and need to be assisted by a surgeon from another speciality (vascular for example) which clearly represents a substantial cost and an unnecessary mobilization of skills.

FR-A-2 858 546 describes prostheses made in two parts which can be implanted posteriorly. But both parts of the prosthesis have a truncated sphere, the centres of which must be joined to enable the prosthesis to function normally. This therefore presents difficulties of accuracy when it is put in place.

In addition, in the case where one of the two parts moves even slightly during the life of the patient, the centre of the two truncated spheres is no longer identical and therefore continued function of the assembly is no longer possible.

Furthermore, this type of prosthesis cannot be installed by posterolateral access (TLIF type).

Moreover, it does not allow shocks to be absorbed.

AU-A-4238202 also describes a prosthesis intended to be implanted posteriorly. But its cylindrical shape does not satisfy standard surgical practice. Furthermore, its stability is questionable. Indeed, without arthrodesis, movements continue throughout the life of the patient, and therefore there is concern that unscrewing could occur over time.

In addition, a prosthesis of this type cannot be implanted posterolaterally. Furthermore, it is difficult to impact in herniated disks.

It would therefore be desirable to have available an intervertebral spacer, capable in particular of deforming according to the demands of anatomical movements, which does not require a particular kinematics and is capable of being installed by posterior or posterolateral access.

Now, after extensive research, the Applicant has developed a one- or two-part prosthesis, capable of being installed by posterior or posterolateral access, remedying these drawbacks.

The invention is presented in its most general aspect, in the form of a multilayer intervertebral spacer comprising two elements intended to be anchored effectively on the vertebral plates. It must be remembered here that the inventive devices are intended for the field of posterior spacers, and not for example, the field of anterior spacers. The outcome is that the anterior spacers comprise a single element and generally have a width of 25 to 40 mm, a thickness of 7 to 14 mm and a length of 20 to 30 mm, while the spacers which can be installed by posterior or posterolateral access such as those of the present invention comprising two elements, given that they are separated by the dura mater, generally have a width of 8 to 12 mm, a thickness of 7 to 14 mm and a length of 20 to 26 mm. The bean-shaped spacers of the invention, which are not used in pairs, have a width of approximately 10 mm, a thickness of 7 to 14 mm and a curvilinear length of approximately 25 to 40 mm.

More precisely, the subject of the present application is a multilayer intervertebral spacer, characterized in that it comprises at least three superimposed layers including a lower layer, an upper layer, and at least one intermediate layer, the at least one intermediate layer being made of a different and less rigid material than that of the lower and upper layers, and in that it has dimensions suitable for being installed by posterior or posterolateral access.

Even more precisely, the subject of the present application is a multilayer intervertebral spacer, comprising at least three superimposed layers including a lower layer, an upper layer, and at least one intermediate layer, the at least one intermediate layer being made of a different and less rigid material than that of the lower and upper layers, characterized in that, viewed from above, it has the general shape of a kidney bean.

An intervertebral spacer is by its nature intended to be inserted between two vertebrae. By convention therefore, the layer situated towards the head of a standing individual fitted with the device will be termed the “upper” layer. The other terms having a directional meaning such as “front” and “back” or “high” and “low” also refer to the orientation of the cage when it is implanted in the vertebral column to be fitted.

In preferred conditions of use of the invention, a multilayer intervertebral spacer as above comprises a single intermediate layer.

The at least one intermediate layer can for example be made of elastic material, preferably of elastomer plastic material, particularly of plastic silicone polyaddition or polycondensation plastic material or polyurethane plastic material and quite particularly of polyurethane elastomer as marketed by the company POLY MEDICA INDUSTRIES, Inc (Massachusetts) under the name Chronoflex®. An intermediate layer can be made of a single material or a mixture of the two materials such as a mixture of silicone plastic material and polyurethane.

In other preferred conditions of use of the invention, the at least one intermediate layer comprises moreover one or more lumina distributed regularly or irregularly in said intermediate layer. Thus there is the possibility of having different types of shock absorbency suited to the sought response.

The at least one intermediate layer, of a suitable thickness, is capable of reproducing the articulation movements. For example, if a man bends forwards, the disk situated between L4 and L5 must be compressed by approximately 2 mm at the front; if he bends towards his right side, this disk must be compressed in its right part by approximately 1.5 mm. The elastic piece will thus have the height necessary to allow the preferred maximum ranges required by the anatomical movements. It is also possible for the elastic piece to have the height necessary to allow at least 50%, advantageously at least 70%, particularly at least 80%, quite particularly at least 90% of the maximum ranges required by the anatomical movements.

The at least one intermediate layer can have a thickness of 2 to 12 mm, preferably 3 to 11 mm, in particular 4 to 10 mm, quite particularly 5 to 9 mm.

In the present application and in the following, the stiffness of the material(s) of the at least one intermediate layer is expressed by their hardness.

The hardness of the material(s) of the at least one intermediate layer can for example be comprised between 40 Shore A and 60 Shore D, preferably comprised between 50 Shore A and 55 Shore D, in particular comprised between 65 Shore A and 50 Shore A, particularly comprised between 25 SHD and 45 Shore D, quite particularly comprised between 30 Shore D and 40 Shore D.

The choice of thickness can be combined with the choice of hardness of the material(s) of the at least one intermediate layer and the choice of the shape and size of the lumen (or lumina) in order to obtain the desired shock-absorbency effect.

The at least one intermediate layer can be split through its thickness and receive an insert, in particular made of an elastic material other than the one constituting said at least one intermediate layer. This insert can optionally emerge to the outside, thus constituting an additional intermediate layer. The elastic material of the insert is usually more flexible (or on the contrary, more rigid) than the one constituting said at least one intermediate layer.

The lower layer and the upper layer form plates which are made of hard material and can preferably be made of titanium and advantageously provided with a surface coating, in particular of porous titanium, alone or together with hydroxyapatite. In a standard fashion, their external surface is preferably corrugated or serrated in order to give an initial and long-term stability to an intervertebral spacer according to the invention.

In further preferred conditions of use of the invention, the side of the multilayer intervertebral spacer to be inserted firstly between two vertebrae in vertical cross-section is tapered or spindle-shaped, for example cone- or bullet-shaped.

In still other preferred conditions of use of the invention, the multilayer intervertebral spacer above is generally approximately rectangular parallelepiped-shaped, and in this case is used in pairs, or viewed from above, kidney bean-shaped.

A multilayer intervertebral spacer which is the subject of the present invention can in particular be made by moulding between the plates forming the lower layer and the upper layer internally provided with roughness patches.

A multilayer intervertebral spacer which is the subject of the present invention has useful properties and qualities.

Due to its elastic intermediate layer, it can tolerate imprecise positioning and deforms according to the demands of anatomical movements.

A prosthesis in which there is a contact between two spherical surfaces will generate pure rotational movements. This system therefore imposes the kinematics of a ball and socket joint. During natural movements, the disks perform random movements combining rotation and translation. The presence of an elastic material in the intervertebral spacers of the present invention makes it possible for the spacers to deform as the body demands, without imposing a fixed trajectory. The intervertebral spacers of the present invention do not impose any particular kinematics.

Furthermore, they allow shock-absorbency.

A generally parallelepiped-shaped intervertebral spacer of the present invention has a shape equivalent to that of the cages to be impacted. In this ease two spacers are placed on each side of the dura mater. Such a spacer can be positioned posteriorly according to the same technique as that used for impacted lumbar cages.

An intervertebral spacer of the present invention having the general shape, viewed from above, of a kidney bean, can be positioned posterolaterally according to the same technique as the positioning of kidney bean-shaped cages (TLIF). In this case only a single spacer is put in place.

The bullet shape of the entry zone of a preferred intervertebral spacer of the present invention facilitates the positioning between squeezed disks.

The external structure of the lower and upper layer, like the raised designs produced on the zones in contact with the vertebral plateaux and/or a coating of porous titanium with or without hydroxyapatite coating, gives an intervertebral spacer of the present invention an excellent immediate and long term stability.

An intervertebral spacer of the present invention provided with lumina of different shapes and dimensions and/or using materials of different elasticity to make up the intermediate layer, makes it possible to obtain different types of shock-absorbency.

These properties are illustrated below in the experimental part. They support the use of the above described spacers, in a lumbar arthroplasty by disk prosthesis.

Thus finally, a subject of the present application is a method for lumbar arthroplasty by disk prosthesis in which at least one multilayer intervertebral spacer as above, is inserted between two vertebrae of an individual requiring a lumbar arthroplasty by disk prosthesis.

The invention will be better understood on reference to the attached drawings in which

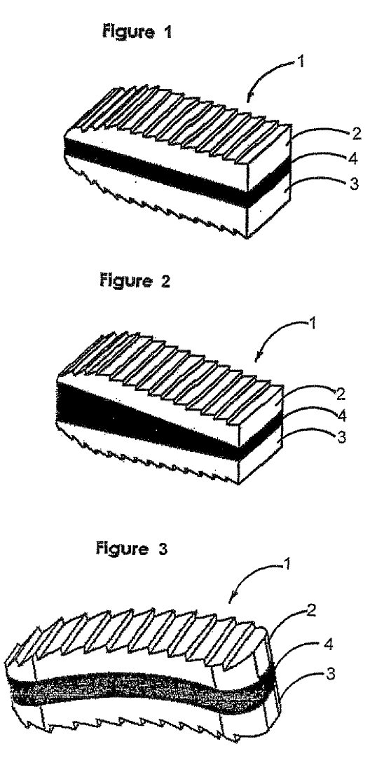

FIGS. 1, 2, 3 and 4 show perspective views of an intervertebral spacer according to the present invention;

FIG. 5 is a top view of a spacer according to the present invention in the shape of a kidney bean of the type shown in FIG. 3, in its final position of installation between two vertebrae;

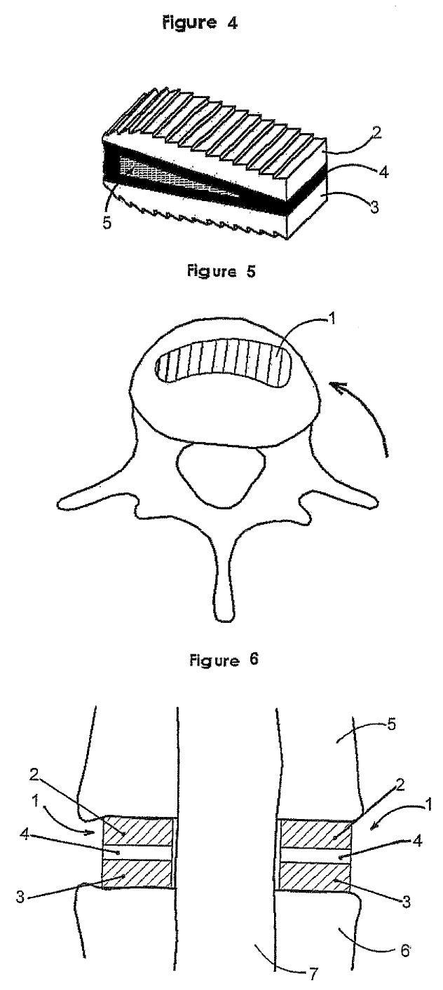

FIG. 6 shows in front cross-section two spacers according to the present invention of the type shown in FIGS. 1, 2 and 4, also installed between two vertebrae, and

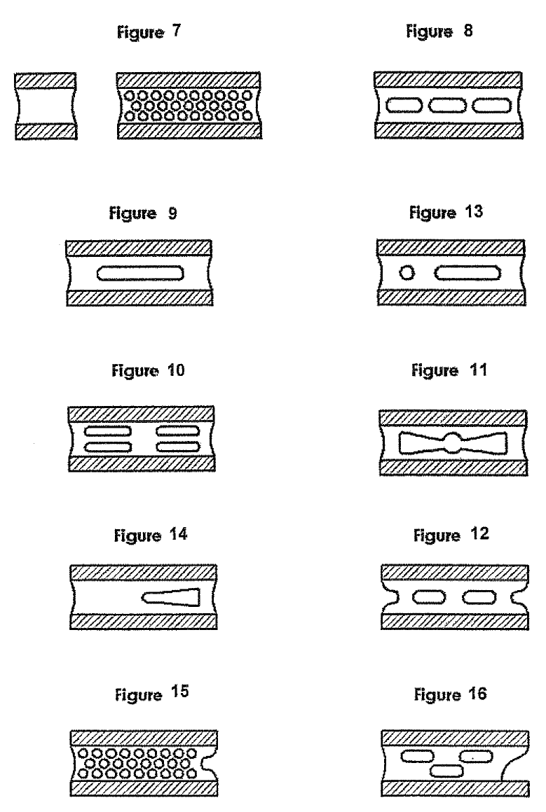

FIGS. 7 to 16 represent, in vertical cross-section, spacers according to the present invention provided with different types of lumen.

In FIG. 1, it is noted that the intervertebral spacer 1 shown comprises three layers, namely an upper layer 2 constituted of a titanium plate with a corrugated surface, a lower layer 3, of the same structure, and an intermediate layer 4 constituted, in the model shown, of polyurethane of 35 Shore D hardness (polyurethane elastomer marketed by the company POLY MEDICA INDUSTRIES, Inc (Massachusetts) under the name Chronoflex®).

It can be seen that the general shape of the intervertebral spacer 1 is that of a rectangular parallelepiped, one of the ends of which, depicted on the left of the diagram, is however spindle-shaped. Such a shape allows easier insertion between two squeezed vertebrae.

In the model shown here, the thickness of the intermediate layer 4 is constant over the entire structure of the intervertebral spacer.

The upper 2 and lower 3 titanium plates become progressively thinner, in order to obtain the desired spindle shape.

In FIG. 2, substantially the same features can be seen as in FIG. 1, however with an intermediate layer 4 which does not have a constant thickness, but which becomes thicker as it comes closer to the spindle-shaped end.

The spacers shown in FIGS. 1 and 2 are used in pairs, as will be seen below in FIG. 6.

The model shown in FIG. 3, on the other hand, is used singly, as shown in FIG. 5. In the intervertebral spacer shown in this FIG. 3, substantially the same features can be seen as in FIG. 1, but nevertheless its general shape is not that of a rectangular parallelepiped, but, viewed from above, a kidney bean shape.

The model shown in FIG. 4 is a variant of the one shown in FIG. 2. But the intermediate layer 4 is composed of two different and separate materials. An insert 5 is in fact made of a material different from the one constituting the intermediate layer 4 in contact with the titanium plates 2 and 3. In a first version, the insert 5 was made with a more rigid plastic material. In another variant produced, the insert is, on the contrary, made of a less rigid material. And in a third variant, the insert is a titanium insert.

In the model shown, the insert 5 is directly accessible from the sides, but does not emerge to the outside at the part of the intervertebral spacer 1 intended to be introduced first between two vertebrae, on the left of the figure.

In FIG. 5, an intervertebral spacer 1 of the type represented FIG. 3 can be seen, installed in position. The arrow shows the direction of introduction of the intervertebral spacer 1 between two vertebrae by the surgeon.

FIG. 6 shows a front vertical cross-section of two spacers of the type shown in FIG. 1, installed between two adjacent vertebrae 5, 6. In the centre of the drawing the dura mater 7 is shown.

FIGS. 7 to 16 show spacers according to the invention comprising various lumina. These lumina are multiple and distributed approximately symmetrically in the spacer for the spacers of FIGS. 7 to 11.

In the spacer shown in FIG. 12, the longitudinal ends of the spacer are substantially without the intermediate layer, due to the presence of a lumen opening to the outside. The same applies to FIGS. 15 and 16.

With the spacers shown in FIGS. 7 and 8, a homogeneous deformation of the spacer is obtained.

With the spacer shown in FIG. 9, the deformation at the centre is greater, which increases the shock-absorbency.

With the spacer show in FIG. 10, the two superimposed lumina increase the deformation for an equal load.

With the spacer shown in FIG. 11, the large lumina on each side increase the deformation capacity under flexion and extension.

With the spacer shown in FIG. 12, the recessing of the material at the ends further promotes the deformation capacity under flexion and extension.

With the spacer shown in FIG. 13, the lumina are arranged in an asymmetrical fashion in order to promote the flexion or extension.

With the spacer shown in FIG. 14, the lumen installed on one side only promotes flexion or extension. If the lumen is placed posteriorly, extension is promoted, if the lumen is placed anteriorly, flexion is promoted.

With the spacers shown in FIGS. 15 and 16, homogenous deformation and recessing of one side only are combined to promote flexion or extension. If the recess is positioned posteriorly, extension is promoted, if it is positioned anteriorly, flexion is promoted.

1. A multilayer intervertebral spacer (1), characterized in that it comprises at least three superimposed layers including a lower layer (3), an upper layer (2), and at least one intermediate layer (4), the at least one intermediate layer (4) being made of a different and less rigid material than that of the lower (3) and upper (2) layers, and in that it has dimensions suitable for being installed by posterior or posterolateral access.

2. A multilayer intervertebral spacer according to claim 1, characterized in that, viewed from above, it has the general shape of a kidney bean.

3. A multilayer intervertebral spacer according to claim 1, characterized in that the side of the multilayer intervertebral spacer (1) to be inserted first between two vertebrae has a tapered or spindle shape in vertical cross-section.

4. A multilayer intervertebral spacer according to claim 1, characterized in that the at least one intermediate layer (4) comprises moreover one or more lumina distributed regularly or irregularly in said intermediate layer (4).

5. A multilayer intervertebral spacer according to claim 1, characterized in that it comprises a single intermediate layer (4).

6. A multilayer intervertebral spacer according to claim 1, characterized in that the at least one intermediate layer (4) is made of elastic material, such as elastomer plastic material, such as polyaddition or polycondensation silicone plastic material or polyurethane plastic material.

7. A multilayer intervertebral spacer according to claim 1, characterized in that the at least one intermediate layer (4) has a thickness of 3 to 11 mm.

8. A multilayer intervertebral spacer according to claim 1, characterized in that the hardness of the material(s) of the at least one intermediate layer (4) is comprised between 50 Shore A and 55 Shore D.

9. A multilayer intervertebral spacer according to claim 1, characterized in that the at least one intermediate layer (4) is split in its thickness and receives an insert (5).

10. A multilayer intervertebral spacer according to claim 2, characterized in that the side of the multilayer intervertebral spacer (1) to be inserted first between two vertebrae has a tapered or spindle shape in vertical cross-section.

11. A multilayer intervertebral spacer according to claim 2, characterized in that the at least one intermediate layer (4) comprises moreover one or more lumina distributed regularly or irregularly in said intermediate layer (4).

12. A multilayer intervertebral spacer according to claim 2, characterized in that it comprises a single intermediate layer (4).

13. A multilayer intervertebral spacer according to claim 2, characterized in that the at least one intermediate layer (4) is made of elastic material, such as elastomer plastic material, such as polyaddition or polycondensation silicone plastic material or polyurethane plastic material.

14. A multilayer intervertebral spacer according to claim 2, characterized in that the at least one intermediate layer (4) has a thickness of 3 to 11 mm.

15. A multilayer intervertebral spacer according to claim 2, characterized in that the hardness of the material(s) of the at least one intermediate layer (4) is comprised between Shore A and 55 Shore D.

16. A multilayer intervertebral spacer according to claim 2, characterized in that the at least one intermediate layer (4) is split in its thickness and receives an insert (5).
